(19) United States
(12) Patent Application Publication
(10) Pub. No.: US 2015/0297523 A1
(43) Pub. Date: Oct. 22, 2015

(54) PHARMACEUTICAL COMPOSITION COMPRISING BENDAMUSTINE

(71) Applicants: Ulhas Dhuppad, Nashik (IN); Dattatray Patil, Nashik (IN)

(72) Inventors: Ulhas Dhuppad, Nashik (IN); Dattatray Patil, Nashik (IN)

(73) Assignee: Glenmark Pharmaceuticals Limited, Mumbai (IN)

(21) Appl. No.: 14/291,799

(22) Filed: May 30, 2014

(30) Foreign Application Priority Data
Apr. 16, 2014 (IN) 1372/MUM/2014

(51) Int. Cl. A61K 9/19; A61K 47/10; A61K 31/4184

(52) U.S. Cl. CPC A61K 9/19; A61K 31/4184; A61K 47/10

(57) ABSTRACT

The present invention relates to a pre-lyophilized composition comprising bendamustine or its pharmaceutically acceptable salt, a pharmaceutically acceptable carrier, an organic solvent and water. Furthermore, the present invention provides a stable pharmaceutical composition comprising bendamustine hydrochloride prepared from such pre-lyophilized composition, a process for preparing such composition; and its use in the treatment of cancer.

### PRIORITY

This application claims priority to Indian Application No. 1372/MUM/2014 filed on Apr. 16, 2014, and entitled “PHARMACEUTICAL COMPOSITION COMPRISING BENDAMUSTINE”, the contents of which are incorporated herein by reference.

### FIELD OF THE INVENTION

The present patent application relates to a pharmaceutical composition comprising bendamustine or its pharmaceutically acceptable salt. More particularly, the present patent application relates to a stable pharmaceutical composition for parenteral administration comprising bendamustine or its pharmaceutically acceptable salt and a pharmaceutically acceptable carrier; a process for preparing such composition; and its use in the treatment of cancer.

### BACKGROUND OF THE INVENTION

Bendamustine was first synthesized in 1963 by Ozegowski and Krebs. It was available in Germany from the early 1970's to 1992, under the brand name CYTOSTATAN®. Since 1993, bendamustine has been marketed under the trade name RIBOMUSTIN® in Germany by Mundipharma International Corporation Limited. It has been widely used as a single-agent or in combination with other anti-cancer agents for the treatment of indolent non-Hodgkin's lymphoma (NHL), multiple myeloma, and chronic lymphocyte leukemia (CLL).

Bendamustine has been reported as a highly unstable compound due to its degradation in aqueous solution. In order to prevent its degradation tendency, bendamustine has been supplied as a lyophilized product. In particular, Ribomustin® is a lyophilized formulation which contains bendamustine HCl and mannitol in a sterile lyophilized form as a white powder for intravenous use following reconstitution.

In the US, bendamustine is sold under the trade name TREANDA® and approved for treatment of chronic lymphocytic leukemia (CLL) and indolent B-cell non-Hodgkin's lymphoma (NHL) that has progressed during or within six months of treatment with rituximab or a rituximab-containing regimen. TREANDA® contains bendamustine hydrochloride and mannitol, which is supplied as a sterile non-pyrogenic white to off-white powder for intravenous use following reconstitution. Treanda® is formulated as lyophilized powder for injection with 25 mg bendamustine and 100 mg bendamustine.

U.S. Patent Publication No. 2006/0159713 discloses a lyophilized formulation of bendamustine. This lyophilized formulation of bendamustine is prepared by lyophilizing a pre-lyophilizing solution comprising bendamustine, tertiary butyl alcohol, mannitol and water.

PCT publication No WO/2012/103226 discloses a lyophilized bendamustine formulations prepared by using of an organic solvent with water, as pre-lyophilization bulk solvent systems, wherein such organic solvents do not include tertiary-butanol or ethanol.

Thus, there is a need to develop a stable lyophilized composition of bendamustine.

### SUMMARY OF THE INVENTION

The present invention relates to a stable pharmaceutical composition comprising bendamustine or its pharmaceutically acceptable salt.

The inventors of present invention have unexpectedly found that the formation of bendamustine ethyl ester impurity is controlled when bendamustine is dissolved in a particular range of concentration in a specific solvent before lyophilization.

The present invention relates to a pre-lyophilized composition comprising bendamustine or its pharmaceutically acceptable salt, a pharmaceutically acceptable carrier, organic solvent and water.

In an embodiment, the organic solvent is selected from group consisting of ethanol, n-propanol, n-butanol, acetontirile, dimethylsulfoxide, acetone or combination thereof.

In an embodiment, the pre-lyophilized composition comprises bendamustine hydrochloride, pharmaceutically acceptable carrier, ethanol and water.

In an embodiment, the pre-lyophilized composition comprises bendamustine hydrochloride, mannitol, ethanol and water.

In an embodiment, the pre-lyophilized composition comprises bendamustine hydrochloride, mannitol, n-butanol and water.

In an embodiment, the pre-lyophilized composition comprises bendamustine hydrochloride, mannitol, acetone, and water.

In an embodiment, the pre-lyophilized composition comprises bendamustine hydrochloride, sucrose, ethanol and water.

In an embodiment, the pre-lyophilized composition comprises bendamustine hydrochloride, sorbitol, n-propanol and water.

In an embodiment, the pre-lyophilized composition comprises from about 5 mg/ml to about 40 mg/ml bendamustine, from about 20 mg/ml to about 50 mg/ml mannitol and from about 10% v/v to about 30% v/v ethanol.

In an embodiment, the pre-lyophilized composition comprises from about 5 mg/ml to about 40 mg/ml bendamustine, from about 20 mg/ml to about 50 mg/ml mannitol and from about 10% v/v to about 30% v/v n-butanol.

In an embodiment, the pre-lyophilized composition comprises from about 5 mg/ml to about 40 mg/ml bendamustine, from about 20 mg/ml to about 50 mg/ml mannitol and from about 10% v/v to about 30% v/v acetone.

In an embodiment, the pre-lyophilized composition comprises from about 5 mg/ml to about 40 mg/ml bendamustine, from about 20 mg/ml to about 50 mg/ml sucrose and from about 10% v/v to about 30% v/v ethanol.

In an embodiment, the pre-lyophilized composition comprises from about 5 mg/ml to about 40 mg/ml bendamustine, from about 20 mg/ml to about 50 mg/ml sorbitol and from about 10% v/v to about 30% v/v n-propanol.

In an embodiment, the pre-lyophilized composition comprises from about 10 mg/ml to about 30 mg/ml bendamustine, from about 25 mg/ml to about 40 mg/mil mannitol and from about 15% v/v to about 25% v/v ethanol.

In a preferred embodiment, the pre-lyophilized composition comprises about 20 mg/ml bendamustine hydrochloride, about 34 mg/ml mannitol and about 20% v/v ethanol.

In another embodiment of the present invention, the pre-lyophilized composition can be in the form of a solution or dispersion which is suitable for lyophilization. Preferably, the pre-lyophilized composition is in the form of solution.

In another embodiment of the present invention, the pH of the pre-lyophilized composition ranges from about 2 to 5, or preferably from about 2.5 to 4.5.

The present invention also relates to a stable pharmaceutical composition obtained after lyophilizing the pre-lyophilized composition described herein. The stable pharmaceutical composition is a lyophilized product comprises bendamustine and pharmaceutically acceptable carrier.

The present invention also relates to a process of preparing a stable pharmaceutical composition which is a lyophilized product, said process comprising:

(a) adding a pharmaceutically acceptable carrier into a first vessel containing water for injection (WFI) under continuous stirring to obtain a visibly clear solution (composition I),

(b) dispersing bendamustine hydrochloride in an appropriate quantity of organic solvent in a second vessel under continuous stirring to form a pale yellow suspension, and then adding an appropriate quantity of water for injection (WFI) under continuous stirring to obtain composition II,

(c) filling composition I and composition II into a vial, partially stoppering it (autoclaved and dried) with a rubber stopper and maintaining a temperature between about 2° to about 8° C.; and

(d) loading the partially stoppered vials into a pre cooled chamber of a lyophilizer at −10° C. and closing the chamber for a lyophilization process.

In another embodiment of the present invention, the stable pharmaceutical composition of bendamustine having water content not more than about 5% w/w or preferably not more than about 2% w/w.

In another embodiment of the present invention, the stable pharmaceutical composition of bendamustine having reconstitution time less than about 5 minutes or preferably less than about 3 minutes.

In another embodiment of the present invention, the stable pharmaceutical composition of bendamustine having a pH in the range from about 2.5 to about 4.5.

In another embodiment of the present invention, the stable pharmaceutical composition contains not more than about 1% w/w, or preferably not more than about 0.5% w/w bendamustine hydroxy impurity.

In another embodiment of the present invention, the stable pharmaceutical composition contains not more than about 1% w/w, or preferably not more than about 0.5% w/w bendamustine ethylester impurity.

In another embodiment of the present invention, the stable pharmaceutical composition contains not more than about 2% w/w, or preferably not more than about 1% w/w total impurity.

In another embodiment, the present invention provides a method of treating a medical condition in a patient that involves administering a therapeutically effective amount of a sable pharmaceutical composition of the present invention where the condition is amenable to treatment with said stable pharmaceutical composition. Examples of conditions amenable to treatment with the compositions of the invention include chronic lymphocytic leukemia (CLL), Hodgkin's disease, non-Hodgkin's lymphoma (NHL), multiple myeloma (MM), breast cancer, small cell lung cancer, hyperproliferative disorders, and an autoimmune disease.

### DETAILED DESCRIPTION OF THE INVENTION

### Definitions

The term “bendamustine” refers to the compound called bendamustine, pharmaceutically acceptable salts of bendamustine, isomers, racemates, solvates, complexes and hydrates, anhydrate forms thereof, and any polymorphic or amorphous forms thereof or combinations thereof.

The term “stable pharmaceutical composition” refers to a pharmaceutical composition of bendamustine having sufficient stability to allow storage at a convenient temperature, such as between about 10° C. to about 25° C. for a period of time, such as at least about six months, at least about one year, or at least about 2 years.

Further the term “stable pharmaceutical composition” also refers to a pharmaceutical composition of bendamustine having sufficient stability to allow storage at a temperature, such as between about 35° C. to about 50° C. for a period of time, such as at least about one month or at least about six month.

The term “salt” or “pharmaceutically acceptable salt”, it is meant those salts and esters which are, within the scope of sound medical judgment, suitable for use in contact with the tissues of humans and lower animals without undue toxicity, irritation, and allergic response, commensurate with a reasonable benefit to risk ratio, and effective for their intended use. Representative acid additions salts include the hydrochloride, furoate, hydrobromide, sulphate, bisulphate, acetate, oxalate, valerate, oleate, palmitate, stearate, laurate, borate, benzoate, lactate, phosphate, tosylate, mesylate, citrate, maleate, fumarate, succinate, tartrate, ascorbate, glucoheptonate, lactobionate, and lauryl sulphate salts. Representative alkali or alkaline earth metal salts include the sodium, calcium, potassium and magnesium salts.

The term “pre-lyophilizing composition” as used herein refers to a solution or dispersion wherein bendamustine or its acceptable salt (preferably hydrochloride) is either completely or partially solubilized or dispersed in a solvent composed of aqueous and one or more non-aqueous solvents, more preferably the non-aqueous solvent is ethanol.

The term “lyophilized product” as used herein refers to solid material obtained by lyophilization, i.e., freeze-drying a solution composed of aqueous and one or more non-aqueous solvents, more preferably the non-aqueous solvent is ethanol.

The term “pharmaceutically acceptable carrier” as used herein refers to a substance which is pharmaceutically acceptable and is used to formulate a pharmaceutical composition of bendamustine or its salt. Preferably, a carrier is therapeutically inert material which includes, but is not limited to, stabilizers, bulking agents, buffers, carriers, diluents, vehicles, and solubilizer. The pharmaceutically acceptable carrier useful for the present application includes, but is not limited to, mannitol, sorbitol, lactose, sucrose, maltose, dextrose, trehalose, sodium or potassium phosphate, citric acid and tartaric acid. Other carriers that may be used, if desired, include antioxidants, such as, but not limited to, ascorbic acid, acetylcysteine, cysteine, sodium hydrogen sulfite, butylated hydroxyanisole, butylated hydroxytoluene, alpha-tocopherol acetate, and chelating agents.

As used herein, the term “treating” or “treatment” of a state, disorder or condition mean: (1) inhibiting the state, disorder or condition, i.e., arresting or reducing the development of the disease or at least one clinical or sub-clinical symptom thereof, or (2) relieving the disease, i.e., causing regression of the state, disorder or condition or at least one of its clinical or sub-clinical symptoms. The benefit to a subject to be treated is either statistically significant or at least perceptible to the subject or to the physician.

The term “therapeutically effective amount” as used herein refers to that amount of the compound being administered that will relieve to some extent one or more of the symptoms of the disorder being treated. Further, a therapeutically effective amount can be that amount that increases the life expectancy of a patient afflicted with a terminal disorder.

The present invention relates to a stable pharmaceutical composition comprising bendamustine or its pharmaceutically acceptable salts, isomers, racemates, enantiomers, hydrates, solvates, metabolites, polymorphs, and mixtures thereof.

The present invention relates to a pre-lyophilized composition comprising bendamustine or its pharmaceutically acceptable salt, pharmaceutically acceptable carrier, organic solvent and water.

In an embodiment, the organic solvent is selected from group consisting of ethanol, n-propanol, n-butanol, acetontirile, dimethyl sulfoxide, acetone or combination thereof.

In an embodiment, the pre-lyophilized composition comprises bendamustine, pharmaceutically acceptable carrier, ethanol and water.

In an embodiment, the pre-lyophilized composition comprises bendamustine, mannitol, ethanol and water.

In an embodiment, the pre-lyophilized composition comprises bendamustine, mannitol, n-butanol and water.

In an embodiment, the pre-lyophilized composition comprises bendamustine, mannitol, acetone, and water.

In an embodiment, the pre-lyophilized composition comprises bendamustine, sucrose, ethanol and water.

In an embodiment, the pre-lyophilized composition comprises bendamustine, sorbitol, n-propanol and water.

In an embodiment, the pre-lyophilized composition comprises from about 5 mg/ml to about 40 mg/ml bendamustine, from about 20 mg/ml to about 50 mg/ml mannitol and from about 10% v/v to about 30% v/v ethanol.

In an embodiment, the pre-lyophilized composition comprises from about 5 mg/ml to about 40 mg/ml bendamustine, from about 20 mg/ml to about 50 mg/ml mannitol and from about 10% v/v to about 30% v/v n-butanol.

In an embodiment, the pre-lyophilized composition comprises from about 5 mg/ml to about 40 mg/ml bendamustine, from about 20 mg/ml to about 50 mg/ml mannitol and from about 10% v/v to about 30% v/v acetone.

In an embodiment, the pre-lyophilized composition comprises from about 5 mg/ml to about 40 mg/ml bendamustine, from about 20 mg/ml to about 50 mg/ml sucrose and from about 10% v/v to about 30% v/v ethanol.

In an embodiment, the pre-lyophilized composition comprises from about 5 mg/ml to about 40 mg/ml bendamustine, from about 20 mg/ml to about 50 mg/ml sorbitol and from about 10% v/v to about 30% v/v n-propanol.

In an embodiment, the pre-lyophilized composition comprises about 10 mg/ml to about 30 mg/ml bendamustine, about 25 mg/ml to about 40 mg/ml mannitol and about 15% v/v to about 25% v/v ethanol.

In a preferred embodiment, the pre-lyophilized composition comprises about 20 mg/ml bendamustine hydrochloride, about 34 mg/ml mannitol and about 20% v/v ethanol.

In another embodiment of the present invention, the pre-lyophilized composition can be in the form of a solution or dispersion which is suitable for lyophilization. Preferably, the pre-lyophilized composition is in the form of a solution.

In another embodiment of the present invention, the pH of the pre-lyophilized composition ranges from about 2 to 5, or preferably from about 2.5 to 4.5.

In another embodiment, the pre-lyophilized composition further comprises a pharmaceutical acceptable carrier.

The present invention also relates to a stable pharmaceutical composition obtained after lyophilizing the pre-lyophilized composition described herein. The stable pharmaceutical composition is a lyophilized product comprises bendamustine and pharmaceutically acceptable carrier.

The present invention also relates to a process of preparing a stable pharmaceutical composition which is a lyophilized product, said process comprising:

(a) adding a pharmaceutically acceptable carrier into a first vessel containing water for injection (WFI) under continuous stirring to obtain a visibly clear solution, (composition I),

(b) dispersing bendamustine hydrochloride in an appropriate quantity of organic solvent in a second vessel under continuous stirring to form a pale yellow suspension, and then adding an appropriate quantity of water for injection (WFI) under continuous stirring to obtain composition II,

(c) filling composition I and composition II into a vial, partially stoppering it (autoclaved and dried) with a rubber stopper and maintaining a temperature between about 2° to about 8° C., and

(d) loading the partially stoppered vial into a pre cooled chamber of lyophilizer at a temperature of −10° C. and closing the chamber for a lyophilization process.

In another embodiment of the present invention, a stable pharmaceutical composition of bendamustine is provided having a water content of not more than about 5% w/w or preferably not more than 2% w/w.

In another embodiment of the present invention, a stable pharmaceutical composition of bendamustine is provided having reconstitution time less than about 5 minutes or preferably less than about 3 minutes.

In another embodiment of the present invention, a stable pharmaceutical composition of bendamustine is provided having a pH in the range from about 2.5 to about 4.5.

In another embodiment of the present invention, a stable pharmaceutical composition is provided which contains not more than about 1% w/w, or preferably not more than about 0.5% w/w bendamustine hydroxy impurity.

In another embodiment of the present invention, a stable pharmaceutical composition is provided which contains not more than about 1% w/w, or preferably not more than about 0.5% w/w bendamustine ethylester impurity.

In another embodiment of the present invention, a stable pharmaceutical is provided which contains not more than about 2% w/w, or preferably not more than about 1% w/w total impurity.

Another embodiment of the invention is a method of treating a cancer in a patient that involves administering a therapeutically effective amount of a pharmaceutical composition of the present invention. Examples of cancer conditions amenable to such treatment include chronic lymphocytic leukemia (CLL), Hodgkin's disease, non-Hodgkin's lymphoma (NHL), multiple myeloma (MM), breast cancer, small cell lung cancer, hyperproliferative disorders, and an autoimmune disease.

The pre-lyophilization composition can be sterilized before lyophilization. Sterilization is generally performed by aseptic filtration, e.g., through a 0.22 micron or less filter.

Sterilization of the pre-lyophilized composition can be achieved by other methods known in the art, e.g., filtration, radiation.

In the present invention, the pre-lyophilizing composition is lyophilized after sterilization. The formulation can be effectively and efficiently lyophilized in the containers in which the product is to be marketed, such as, a vial.

Another aspect of the invention is a method for preparing a lyophilized product of bendamustine by lyophilizing the pre-lyophilization composition comprising the steps of:

(i) freezing the pre-lyophilization composition to a temperature below about −40° C., preferably −50° C., to form a frozen solution;

(ii) ramping the frozen solution at or below −40° C., for at least 2 hours to about 4 hours;

(iii) holding the frozen solution at or below −50° C., for about 10 hours to 15 hours;

(iv) ramping the frozen solution to a primary drying temperature between about −50° C. and about −5° C. to form a dried solution;

(v) holding for about 10 to about 60 hours;

(vi) ramping the dried solution to a secondary drying temperature between about 25° C. and about 40° C.; and

(vii) holding for about 5 to about 20 hours to form a bendamustine lyophilized product.

Preferably, the method of lyophilizing the pre-lyophilization composition comprises the following steps—

(i) freezing the pre-lyophilization composition at or below −40° C. to form a frozen solution;

(ii) ramping the frozen solution at or below −40° C. for at least 2 hours;

(iii) holding the frozen solution at about −50° C. for about 10 hours;

(iv) ramping the frozen solution to a primary drying temperature between about −40° C. and about −5° C. to form a dried solution;

(v) holding the dried solution at a primary drying temperature for about 20 to about 40 hours;

(vi) ramping the dried solution to a secondary drying temperature between about 25° C. and about 40° C.; and

holding at a secondary drying temperature for at least 6 hours up to about 12 hours.

It will be understood that one skilled in the art can change the order of the steps and quantities as needed.

The following examples are provided to enable one skilled in the art to practice the invention and are merely illustrative of the invention. The examples should not be read as limiting the scope of the invention.

### Examples 1-5

### Compositions of Bendamustine Hydrochloride for Parenteral Administration

| | Quantity/Unit (Qty per vial in mg) | | | | |
|---|---|---|---|---|---|
| Ingredients | 1 | 2 | 3 | 4 | 5 |
| D-Mannitol | — | 170 | — | 170 | 170 |
| Sorbitol | 170 | — | — | — | — |
| Sucrose | — | — | 170 | — | — |
| Water for Injection¹ | q.s to | q.s to | q.s to | q.s to | q.s to |
| | 3.5 ml | 3.5 ml | 3.5 ml | 3.5 ml | 3.5 ml |
| Bendamustine HCl | 100 | 100 | 100 | 100 | 100 |
| Ethanol¹ | — | 1 ml | 1 ml | — | — |
| n-propanol¹ | 1 ml | — | — | — | — |
| n-butanol¹ | — | — | — | 1 ml | — |
| Acetone¹ | — | — | — | — | 1 ml |
| Water for Injection¹ | q.s to | q.s to | q.s to | q.s to | q.s to |
| | 1.5 ml | 1.5 ml | 1.5 ml | 1.5 ml | 1.5 ml |

q.s.: Quantity sufficient;
¹does not appear in the final product.

### Examples 6-10

### Compositions of Bendamustine Hydrochloride for Parenteral Administration

| | Quantity/Unit (Qty per vial in mg) | | | | |
|---|---|---|---|---|---|
| Ingredients | 6 | 7 | 8 | 9 | 10 |
| D-Mannitol | — | 42.5 | — | 42.5 | 42.5 |
| Sorbitol | 42.5 | — | — | — | — |
| Sucrose | — | — | 42.5 | — | — |
| Water for Injection¹ | q.s to | q.s to | q.s to | q.s to | q.s to |
| | 0.875 ml | 0.875 ml | 0.875 ml | 0.875 ml | 0.875 ml |
| Bendamustine HC1 | 25 | 25 | 25 | 25 | 25 |
| Ethanol¹ | — | 0.25 ml | 0.25 ml | — | — |
| n-propanol¹ | 0.25 ml | — | — | — | — |
| n-butanol¹ | — | — | — | 0.25 ml | — |
| Acetone¹ | — | — | — | — | 0.25 ml |
| Water for Injection¹ | q.s to | q.s to | q.s to | q.s to | q.s to |
| | 0.375 ml | 0.375 ml | 0.375 ml | 0.375 ml | 0.375 ml |

q.s.: Quantity sufficient;
¹does not appear in the final product.

Manufacturing Procedure for Examples 1-10:

1. Mannitol was added to the manufacturing vessel under continuous stirring containing water for injection (WFI) to obtain a visibly clear solution, while adding mannitol in above vessel the stirring was maintained at 800-1000 rotations per minute (RPM), for 5 to 10 minutes for complete dissolution, the volume was made up with WFI (Composition I)

2. Organic solvent was dispensed into another mixing vessel, Bendamustine Hydrochloride was added under continuous stirring, while adding drug in this vessel, the stirring was maintained at 1000 to 1500 RPM, for 3 to 5 minutes to form pale yellow suspension, then WFI was added at room temperature (RT) under continuous stirring, while adding water in this vessel, the stirring was maintained at 1000 to 1500 RPM, for 3 to 5 minutes to form clear pale yellow solution, the volume was made up with WFI and then cooled at about 2° to about 8° C. in Jacketed vessel. (Composition II)

3. Both compositions I and composition II were separately filtered using polyvinylidene fluoride (PVDF) filters for sterilization at about 20 to about 8° C. under nitrogen pressure.

4. After filtration, composition I and composition II were filled into a depyrogenated vial and the vial was partially stoppered (autoclaved and dried) with a rubber stopper. During filling, the temperature was maintained between about 2° to about 8° C.

5. The partially stoppered vial was loaded into a pre cooled chamber of lyophilizer at −10° C. and then the chamber was closed for a lyophilization process.

6. The lyophilizer was operated as per specified lyophilization parameters as given below.

7. After completion of the lyophilization cycle, the vial was stoppered in the presence of nitrogen atmosphere, and sealed and external cleaned of vial.

Lyophilization Cycle:

The lyophilization cycle as used in lyophilizing the pre-lyophilized solution of bendamustine is given below:

1. Freezing:

Start the Freezing cycle as per schedule mentioned below:

a. The shelf temperature was set to −10° C. and was maintained during vial loading.

b. The shelf temperature was set to −40° C. Ramped for 2 hrs.

c. The shelf temperature was set to −50° C. Ramped for 2 hrs with temperature margin and held for 8 hrs.

2. Primary Drying:

The condenser was started and the condenser temperature was maintained between −65° C. to −70° C. Started the primary drying as per schedule mentioned below:

a. Vacuum to 0.900 mbar was applied, and a shelf temp temperature was set to −40° C. Ramped for 2 hrs and held for 2 hrs.

b. Vacuum to 0.800 mbar was applied, and a shelf temp temperature was set to −40° C. Ramped for 1 hr and held for 3 hrs.

c. Vacuum to 0.500 mbar was applied, and a shelf temp temperature was set to −25° C. Ramped for 2 his and held for 4 hrs.

d. Vacuum to 0.200 mbar was applied, and a shelf temp temperature was set to −25° C. Ramped for 2 hrs and held for 8 hrs.

e. Vacuum to 0.200 mbar was applied, and a shelf temp temperature was set to −15° C. Ramped for 2 hrs and held for 6 hrs.

f. Vacuum to 0.200 mbar was applied, and a shelf temp temperature was set to −5° C. Ramped for 2 hrs and held for 6 hrs.

g. Vacuum to 0.200 mbar was applied, and a shelf temperature was set to 10° C. Ramped for 2 hrs and held for 4 hrs.

3. Secondary Drying:

The vacuum below 0.100 mbar was maintained throughout secondary drying cycle.

The secondary drying was started as per schedule mentioned below:

a. The shelf temperature was set to 25° C. Ramped for 2 hrs and held for 2 hrs.

b. The shelf temp was set to 40° C. Ramped for 2 hrs and held for 4 hrs.

### Example 11

### Stability Data of Bendamustine Composition as Per Example 2 (100 mg/Vial) and Example 7 (25 mg/Vial)

Storage Condition: 25° C./60% Relative Humidity

Pack Details:

Example 2—20 ml amber tubular glass vial USP type 1 with chlorobutyl stopper

Example 7—10 ml amber tubular glass vial USP type 1 with chlorobutyl stopper

| | Example 2 Composition | | Example 7 Composition | |
|---|---|---|---|---|
| | (100 mg/vial) | | (25 mg/vial) | |
| Tests | 6 month | 12 month | 6 month | 12 month |
| Description | White | White | White | White |
| | lyophilized | lyophilized | lyophilized | lyophilize |
| | cake | cake | cake | cake |
| Water content (w/w) | 1% | <1% | 1% | 1% |
| Reconstitution time | 2.2 min. | 2.2 min. | 0.2 min. | 0.4 min. |
| pH of reconstituted | 2.8 | 3.0 | 2.8 | 2.6 |
| solution | | | | |
| Impurity Profile (w/w) | 0.03% | 0.05% | 0.05% | 0.17% |
| N-alkylated | | | | |
| Monohydroxy | 0.18% | 0.21% | 0.19% | 0.19% |
| Ethylester | <0.1% | — | <0.1% | — |
| Dimer | 0.21% | 0.37% | 0.24% | 0.26% |
| Any unspecified | 0.03% | 0.05% | 0.06% | 0.10% |
| impurity | | | | |
| Total Impurity | 0.51% | 0.84% | 0.59% | 0.99% |
| Assay (w/w) | 97.3% | 96.4% | 96.9% | 93.0% |

### Example 12

### Stability Data of Bendamustine Composition as Per Example 2 (100 mg/Vial) and Example 7 (25 mg/Vial)

Storage Condition: 40° C./75% Relative Humidity

Pack Details:

Example 2—20 ml amber tubular glass vial USP type 1 with chlorobutyl stopper

Example 7—10 ml amber tubular glass vial USP type 1 with chlorobutyl stopper

| | Example 2 Composition | | Example 7 Composition | |
|---|---|---|---|---|
| | (100 mg/vial) | | (25 mg/vial) | |
| Tests | 1 month | 3 month | 1 month | 3 month |
| Description | White | White | White | White |
| | lyophilized | lyophilized | lyophilized | lyophilized |
| | cake | cake | cake | cake |
| Water content (w/w) | 1% | 1% | 2% | 1% |
| Reconstitution time | 1.2 min. | 2.2 min. | 0.2 min. | 0.6 min |
| pH of reconstituted | 3.0 | 3.1 | 3.3 | 3.0 |
| solution | | | | |
| Impurity Profile (w/w) | 0.04% | 0.03% | 0.05% | 0.04% |
| N-alkylated | | | | |
| Monohydroxy | 0.15% | 0.10% | 0.16% | 0.14% |
| Ethylester | <0.1% | <0.1% | 0.1% | 0.1% |
| Dimer | 0.24% | 0.22% | 0.28% | 0.25% |
| Any unspecified | 0.09% | 0.04% | 0.10% | 0.06% |
| impurity | | | | |
| Total impurity | 0.63% | 0.52% | 0.71% | 0.68% |
| Assay (w/w) | 96.9% | 96.9% | 98.3% | 95.1% |

Although the invention herein has been described with reference to particular embodiments, it is to be understood that these embodiments are merely illustrative of the principles and application of the present invention. It is therefore to be understood that numerous modifications may be made to the illustrative embodiments and that other arrangements may be devised without departing from the spirit and scope of the present invention as described above.

All publications, patents, and patent applications cited in this application are herein incorporated by reference to the same extent as if each individual publication, patent, or patent application was specifically and individually indicated to be incorporated herein by reference.

What is claimed is:

1. A pre-lyophilized composition comprising bendamustine or its pharmaceutically acceptable salt, a pharmaceutically acceptable carrier, an organic solvent and water.

2. The pre-lyophilized composition according to claim 1, wherein the organic solvent is selected from group consisting of ethanol, n-propanol, n-butanol, acetontirile, dimethylsulfoxide, acetone and combinations thereof.

3. The pre-lyophilized composition according to claim 1, wherein the organic solvent is ethanol.

4. The pre-lyophilized composition according to claim 1, wherein the pharmaceutically acceptable carrier is selected from the group consisting of mannitol, sorbitol, lactose, sucrose, maltose, dextrose, trehalose, sodium phosphate, potassium phosphate, citric acid and tartaric acid.

5. The pre-lyophilized composition according to claim 1, wherein the pharmaceutically acceptable carrier is mannitol.

6. A pre-lyophilized composition according to claim 1, comprising from about 5 mg/ml to about 40 mg/ml bendamustine hydrochloride, from about 20 mg/ml to about 50 mg/ml mannitol and from about 10% v/v to about 30% v/v ethanol.

7. A pre-lyophilized composition comprising about 20 mg/ml bendamustine hydrochloride, about 34 mg/ml mannitol and about 20% v/v ethanol.

8. A process of preparing a stable pharmaceutical composition, said process comprising:
a) adding a pharmaceutically acceptable carrier into a first vessel containing water for injection (WFI) under continuous stirring to obtain a visibly clear solution (composition I);
b) dispersing bendamustine hydrochloride in an appropriate quantity of organic solvent in a second vessel under continuous stirring to form a pale yellow suspension, and then adding an appropriate quantity of water for injection (WFI) under continuous stirring to obtain composition II;
c) filling composition I and composition II into a vial, partially stoppering it with a rubber stopper and maintaining the vial at a temperature between about 2° to about 8° C.; and
d) lyophilizing the vial.

9. The process according to claim 8, wherein the stable pharmaceutical composition comprises water content not more than about 5% w/w.

10. The process according to claim 8, wherein the stable pharmaceutical composition has a reconstitution time less than about 5 minutes.

11. The process according to claim 8, wherein the stable pharmaceutical composition has a pH in the range from about 2.5 to about 4.5.

12. The process according to claim 8, wherein the stable pharmaceutical composition has an ethylester impurity content of not more than about 1% w/w.

13. The process according to claim 8, wherein the stable pharmaceutical composition has a hydroxy impurity content of not more than about 1% w/w.

14. The process according to claim 8, wherein the stable pharmaceutical composition has a total impurity content of not more than about 2% w/w.
